(19) United States
(12) Patent Application Publication
(10) Pub. No.: US 2011/0269196 A1
(43) Pub. Date: Nov. 3, 2011

(54) COENZYME Q10 PRODUCTION USING SPORIDIOBOLUS JOHNSONII

(75) Inventors: Robert Doyle, Syracuse, NY (US); David Dixson, Liverpool, NY (US)

(73) Assignee: SYRACUSE UNIVERSITY, Syracuse, NY (US)

(21) Appl. No.: 13/086,035

(22) Filed: Apr. 13, 2011

Related U.S. Application Data
(60) Provisional application No. 61/323,525, filed on Apr. 13, 2010.

(51) Int. Cl. C12P 7/66; C12N 1/14

(52) U.S. Cl. 435/133; 435/254.1

(57) ABSTRACT

A new isolate of Sporidiobolus johnsonii that is capable of producing over a significant percentage of CoQ₁₀ upon the addition of para-hydroxy benzoic acid in stationary phase, and a method of obtaining CoQ₁₀ from the S. johnsonii isolate which can be scaled up to industrial levels.

### CROSS-REFERENCE TO RELATED APPLICATIONS

The present application claims priority to U.S. Provisional Patent Application No. 61/323,525, filed on Apr. 13, 2010, and entitled “Coenzyme Q10 Production in the Filamentous basidiomycete Sporidiobolus johnsonii,” the content of which is relied upon and incorporated herein by reference in its entirety.

### BACKGROUND OF THE INVENTION

1. Field of the Invention

The present invention relates to method for producing coenzyme Q₁₀ and, more specifically, to a method for producing coenzyme Q₁₀ using a new isolate of Sporidiobolus johnsonii.

2. Description of the Related Art

Ubiquinones, of which coenzyme Q10 (“CoQ₁₀”) is a member, make up a unique class of compounds critical for proper function of the electron transport chain. Via a reversible oxidation-reduction reaction shown in FIG. 1, CoQ₁₀ compounds function as electron mediators between Complex I and II to Complex III in the electron transport chain during aerobic respiration in order to produce a proton gradient, thereby driving the synthesis of ATP. CoQ compounds function as electron mediators between Complex I and II to Complex III in the electron transport chain during aerobic respiration in order to produce a proton gradient, thereby driving the synthesis of ATP. The non-aromatic portion of ubiquinones, namely the isoprenoid tail, functions to solubilize the ubiquinone in the membrane where electron transport occurs. The length of the isoprene tail greatly affects membrane solubility and is species specific. In humans the number of isoprene units is 10, hence the designation “CoQ₁₀.” Other notable ubiquinone examples are CoQ₆ from Saccharomyces cerevisiae and CoQ₈ from Escherichia coli.

In addition to its vital function in ATP production, CoQ₁₀ has been shown to protect against lipid peroxidation in organelle membranes as well as lipoproteins. CoQ₁₀ is the only endogenously produced lipid soluble antioxidant in humans, and low CoQ₁₀ levels have been linked to age related diseases often characterized by decreased energy and susceptibility to reactive oxygen species, such as Alzheimer's disease, Parkinson's disease, and cerebral ataxia. Fortunately, oral supplementation of CoQ₁₀ has been shown to increase serum CoQ₁₀ levels, which alleviates many of the symptoms associated with these diseases. As a result, there is an ever-increasing demand for CoQ₁₀.

Chemical synthesis of CoQ₁₀ can be accomplished using the starting materials trimethoxy toluene and solanesol, as shown in FIG. 2. Using traditional methods, the trimethoxy toluene requires five steps in 76% yield to generate a benzyl chloride intermediate and the solanesol requires two steps in 76% yield to generate vinyl alane. Coupling of these two intermediates provides CoQ₁₀ in approximately 86% yield. This results in a 50% yield from the two starting materials shown. As is immediately apparent, this low yield is cost-prohibitive on a commercial scale.

As a result, biosynthetic production of CoQ₁₀ has been the major focus in the industrial setting. Commercially viable CoQ₁₀ production has been achieved using bacterial species such as Agrobacterium tumefaciens and Rhodobacter sphaeroides producing 1.18% and 1.7% CoQ₁₀ by dry cell weight (respectively). These are the two most significant specific producers of CoQ₁₀ reported to date.

The biosynthesis of CoQ₁₀ can be divided into three parts: (1) production of the aromatic group; (2) production of the isoprene tail; and (3) covalent attachment of the two and subsequent modification. The aromatic precursor, para-hydroxy benzoic acid (“HBA”), is produced from the shikimate pathway. The biosynthesis of the decaprenyl tail starts with formation of the individual isoprene units through the mevalonate or non-mevalonate pathway. Isoprene units are then condensed until chain elongation termination, an event determined by the size of the hydrophobic cavity in the species specific polyprenyl pyrophosphate synthase enzyme, which is species specific. The aromatic group and isoprene tail are covalently attached followed by modification of the aromatic portion resulting in CoQ₁₀.

Genetic engineering efforts aimed at increasing CoQ₁₀ production have most often been attempted in E. coli, which naturally produces CoQ₈. This actual CoQ₈ production means that 1) the CoQ₈ must be purified out from the CoQ₁₀ target, and 2) available isoprene precursors will be used for CoQ₈ and CoQ₁₀ production, unless the wild type octaprenyl diphosphate synthase gene is knocked out. For example, the Gluconobacter suboxydans decaprenyl diphosphate synthase (dpps) gene was expressed in E. coli producing 0.45 mg CoQ₁₀/g DCW, however only about 50% of the total isoprenoid quinone content was CoQ₁₀ with the rest being CoQ₈ and CoQ₉.

The major focus on genetically engineering E. coli for CoQ₁₀ production is to increase the flux through the non-mevalonate isoprenoid pathway, specifically at the rate limiting 1-deoxy-D-xylulose synthase (Dxs) enzyme responsible for the condensation of pyruvate and glyceraldehyde-3-phosphate forming the five carbon unit. Additionally, the E. coli must contain a decaprenyl pyrophosphate synthase gene from a different organism as E. coli does not naturally produce CoQ₁₀. This has recently been accomplished utilizing the dpps gene from Agrobacterium tumefaciens and overexpression of the dxs gene from Pseudomonas aeruginosa as well as deletion of the ispB gene responsible for production of the octaprenyl chain. This provided an increase in specific CoQ₁₀ production from 0.55 mg CoQ₁₀/g DCW to 1.40 mg CoQ₁₀/g DCW. Expressing an entire foreign mevalonate pathway from Streptococcus pneumoniae and decaprenyl pyrophosphate synthase from A. tumefaciens in E. coli increased the specific CoQ₁₀ yield from 0.3 mg CoQ₁₀/g DCW to 2.4 mg CoQ₁₀/g DCW. Various other strategies have been used to increase CoQ₁₀ production in E. coli, but with less success.

This lack of ability to increase specific CoQ₁₀ production through genetically overexpressing biosynthetic enzymes may be a result of the biosynthetic complex necessary for CoQ10 biosynthesis. This biosynthetic complex is theorized to include a variety of enzymatic and non-enzymatic proteins necessary for supercomplex formation enabling substrate channeling. Overexpression of the initial enzymes may result in a build up of intermediate with no supercomplex to continue the biosynthetic process and leads ultimately to degradation of the unstable intermediate.

In light of these difficulties in genetically engineering a highly significant, specific producers of CoQ₁₀, many are actively searching for new natural producers of CoQ₁₀. The bacterial species with the highest specific CoQ₁₀ production reported to date is Agrobacterium tumefaciens, 11.8 mg CoQ₁₀/g DCW. This was increased from 0.55 mg CoQ₁₀/g DCW by inhibiting the electron transport chain with azide, in addition to general optimization of culture conditions and addition of lactate to stimulate the tricarboxylic acid (TCA) cycle.

Recently, it has been shown that many pigmented yeasts are natural CoQ₁₀ producers, some with naturally high levels of CoQ₁₀. Rhodosporidium sphaerocarpum and Sporobolomyces roseus produce up to 1.84 and 0.72 mg CoQ₁₀/g DCW in benchtop conditions using an enriched media.

### BRIEF SUMMARY OF THE INVENTION

It is therefore a principal object and advantage of the present invention to provide a strain of yeast capable of producing CoQ₁₀ at industrially applicable levels.

In accordance with the foregoing objects and advantages, the present invention provides a new strain of a CoQ₁₀ producing member of the Sporobolomyces family, namely Sporidiobolus johnsonii, or a mutant thereof.

A second aspect of the present invention provides a method of producing CoQ₁₀. The method comprises at least the following steps: (1) providing a microbe, wherein the microbe comprises a Sporidiobolus johnsonii strain; (2) culturing the microbe in the presence of a carbon source; and (3) recovering the CoQ₁₀ from the cells in the culture. In a preferred embodiment, the Sporidiobolus johnsonii strain is the new Sporidiobolus johnsonii isolate described herein, or a mutant thereof.

A third aspect of the present invention provides a method of producing CoQ₁₀. The method comprises at least the following steps: (1) providing a microbe, wherein the microbe comprises a Sporidiobolus johnsonii strain; (2) culturing the microbe in the presence of a carbon source; (3) drying the cultured microbe; (4) lysing the microbe; and (5) recovering the CoQ₁₀ from the cells in the culture. In a preferred embodiment, the lysing step is done using a solvent such as methanol.

A fourth aspect of the present invention provides a method of producing CoQ₁₀. The method comprises at least the following steps: (1) providing a microbe, wherein the microbe comprises a Sporidiobolus johnsonii strain; (2) culturing the microbe in the presence of a carbon source; and (3) recovering the CoQ₁₀ from the cells in the culture, where the step of recovering includes: (i) obtaining an extract of the cultured microbe through one of many methods known to one of ordinary skill in the art; and (ii) purifying the CoQ₁₀ from the extract (including, for example, the flash chromatography described below).

A fifth aspect of the present invention provides a method of producing CoQ₁₀. The method comprises at least the following steps: (1) providing a microbe, wherein the microbe comprises a Sporidiobolus johnsonii strain; (2) culturing the microbe in the presence of a carbon source and a CoQ₁₀ biosynthetic precursor; and (3) recovering the CoQ₁₀ from the cells in the culture. In a preferred embodiment, the CoQ₁₀ biosynthetic precursor is 4-Hydroxybenzoic acid or an equivalent, preferably in any known or predicted effective concentration. For example, the experiments described below use anywhere from 0.0 mg/L to about 100 mg/L.

A sixth aspect of the present invention provides a method of producing CoQ₁₀. The method comprises at least the following steps: (1) providing a microbe, wherein the microbe comprises a Sporidiobolus johnsonii strain (preferably the new isolate described below or a mutant thereof); (2) inoculating a culture containing a carbon source with the microbe; (3) culturing the microbe to encourage growth; (4) adding 4-Hydroxybenzoic acid to the culture at some point after the inoculating step; and (5) recovering the CoQ₁₀ using any method described herein or known to those having ordinary skill in the art. The 4-Hydroxybenzoic acid (also known as para-hydroxy benzoic acid or “HBA”) can be added to the culture as a single addition at a single point in time (a “bolus”), or can be slowly and/or continuously added to the culture over an extended period time, or a combination of the two. For example, there can be an initial bolus of HBA followed by a continuous addition for some period of time, or there can be a gradually-increasing but continuous addition over an extended period of time.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic representation of the structure of Coenzyme Q, where ‘n’ represents the number of isoprene monomers;

FIG. 2 is a schematic representation of the organic synthesis of CoQ₁₀ from the starting materials solanisol and trimethoxy toluene, both of which undergo multiple steps in preparation for coupling to form CoQ₁₀ in a 50% overall yield;

FIG. 3 is a photograph of a two day old S. johnsonii colony on an SjMM agar plate;

FIGS. 4A-C are microscopy images of S. johnsonii cells in: (A) exponential phase; (B) early stationary phase; and (C) late stationary phase;

FIG. 5A is graph depicting the growth profile of S. johnsonii with varying amounts of sucrose (♦=5 g, ▪=10 g, ▴=20 g, =30 g, and ∘=Fermenter 5 g);

FIG. 5B is a graph depicting the Dry Cell Weight (“DCW”) of S. johnsonii cultures as a function of sucrose concentration, wherein values represent an average of three trials with error bars representing one standard deviation;

FIG. 6A is a graph depicting the extraction efficiency of the first extraction using several different solvents, wherein bars represent the total percent mass extracted as a percentage of DCW, and further wherein values represent an average of three trials with error bars representing one standard deviation;

FIG. 6B is a graph depicting the extraction efficiency of hexanes wash of the first extract, wherein bars represent the total percent mass extracted as a percentage of DCW, and further wherein values represent an average of three trials with error bars representing one standard deviation;

FIG. 7A is a graph depicting the APCI-MS negative mode of CoQ₁₀ where MS from sample scraped from TLC plate;

FIG. 7B is a graph depicting the APCI-MS negative mode of CoQ₁₀ where MS of CoQ₁₀ standard matches the MS from the extract;

FIG. 8 is a graph showing ¹H NMR of CoQ₁₀ in CDCl₃ where (top) is a CoQ₁₀ standard and (bottom) is CoQ₁₀ purified from extract match the standard (the peak at 1.55 ppm is water from the CDCl₃ solvent);

FIG. 9 is a graph of ¹H NMR spectra of impurity, with key peaks b, c, and d representing the glycerol unit;

FIG. 10 is a graph of MALDI-MS of the TAG showing [M+Na] at m/z=933 amu;

FIG. 11A is a graph of APCI-MS (negative) of closely eluting impurity with a DCL voltage of −7 V showing prominent fragments at m/z=279, 508, 529, and 644 amu;

FIG. 11B is a graph of APCI-MS (negative) of closely eluting impurity with a DCL voltage of −5 V cone voltage showing prominent fragments at m/z=279, 508, 529, and 644 amu;

FIG. 12 is schematic representation of fragmentation during APCI-MS resulting in the ions detected at 645, 529, 508 and 279 amu;

FIG. 13 is a UV trace at 210 nm and 275 nm of the crude extract using a Teledyne Redi-Sep Rf Gold 3 μm silica cartridge showing that the method is successful at separating the TAG and CoQ₁₀;

FIG. 14 is a schematic representation of the biosynthesis of CoQ₁₀, where Shikimate forms the aromatic group para-Hydroxy benzoic acid (“HBA”), while dimethyl allyl phosphate forms the isoprene chain and these are coupled and further modified to produce CoQ₁₀;

FIG. 15A is a graph of the growth profile of varying concentrations of HBA added at 60 hours, wherein values represent an average of three trials with error bars representing one standard deviation;

FIG. 15B is a graph of DCW as a function of HBA added which shows no noticeable affect on DCW at the concentrations tested, wherein values represent an average of three trials with error bars representing one standard deviation;

FIG. 16 is a graph of specific CoQ₁₀ production in S. johnsonii as a function of HBA concentration, wherein values represent an average of three trials with error bars representing one standard deviation;

FIG. 17 is a growth profile of S. johnsonii;

FIG. 18 is a growth profile of a 14 L fermentation of S. johnsonii with a 25 mg/L HBA injection;

FIG. 19 is a growth profile of a 14 L fermentation of S. johnsonii with a bolus addition of 0.085 g/L at 78 hrs followed by the steady addition of 0.02 g HBA/hr;

FIG. 20 is a growth profile of a 14 L fermentation of S. johnsonii with a steady HBA addition of 0.10 g/hr starting at 73 hrs and increasing to 0.15 g/L at 96 hrs;

FIG. 21 is a growth profile of a 14 L fermentation of S. johnsonii with a steady HBA addition starting at 76 hrs;

FIG. 22A depicts ¹H NMR of the crude extract from S. johnsonii cells dried under a cyclone drying method; and

FIG. 22B depicts ¹H NMR of the crude extract from S. johnsonii cells dried under a blow down drying method.

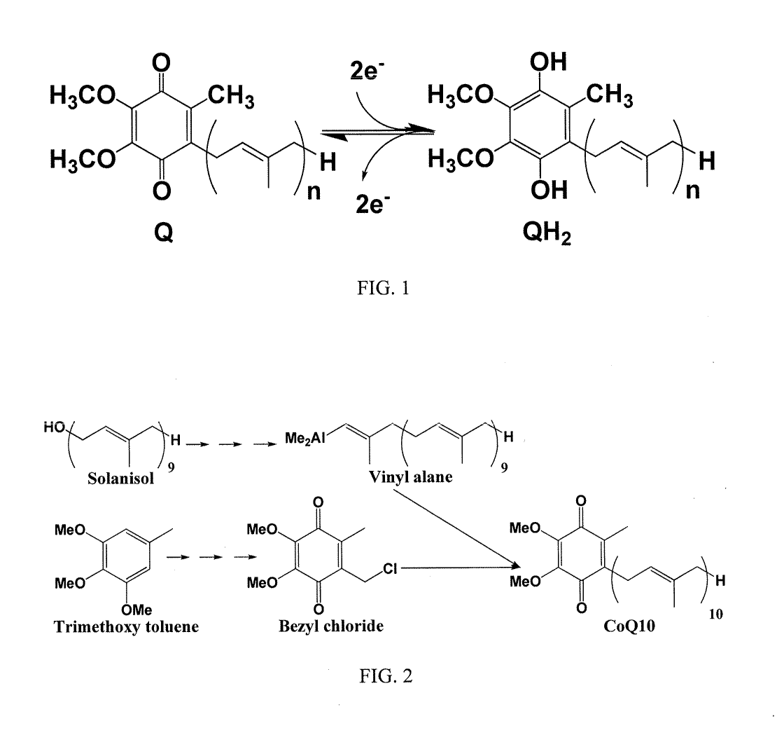

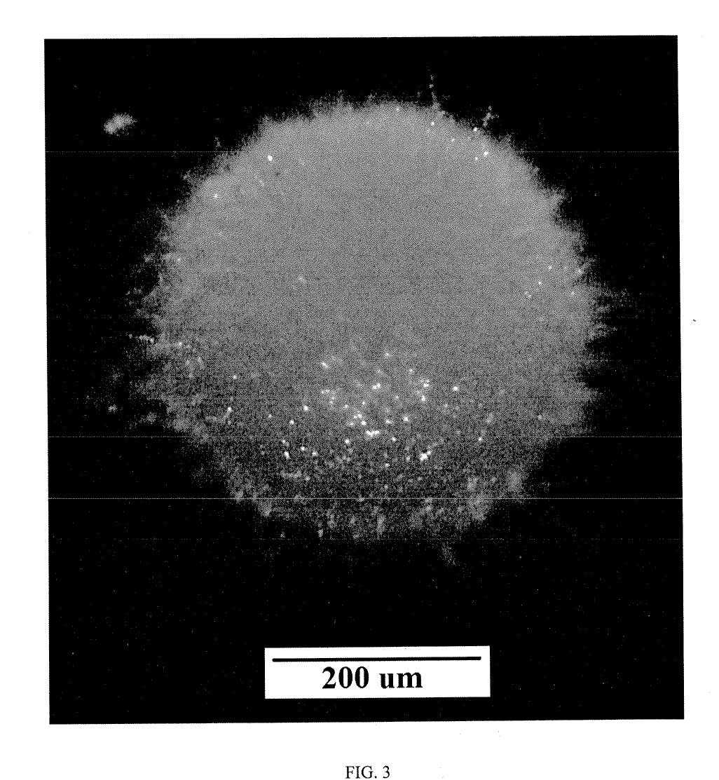

### DETAILED DESCRIPTION OF THE INVENTION

Referring now to the drawings, wherein like reference numerals refer to like parts throughout, the present invention comprises a new isolate of Sporidiobolus johnsonii that is capable of producing over 1% CoQ₁₀ upon the addition of para-hydroxy benzoic acid in stationary phase. The present invention further comprises additional downstream processes including improved extraction methods and purification including separating and identifying a major CoQ₁₀ co-eluting impurity.

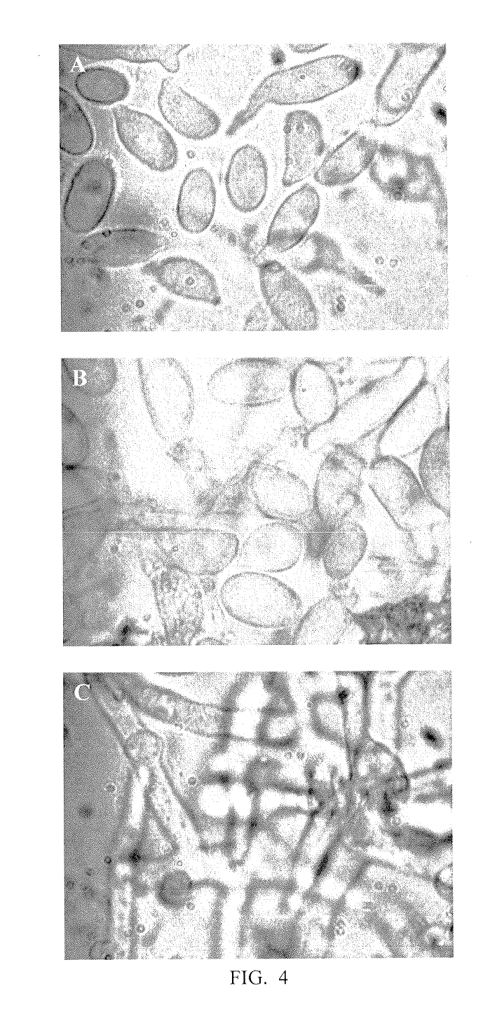

The yeast reported in this paper was initially collected by Daniel DeBrouse (BioSym Technologies of Iowa) from soil around Oklahoma City, Okla., USA and designated Sj0801. The species was then purified to homogeneity by successive plating on enriched media agar (see media recipes herein). Each stage of species purification consisted of selecting six colonies to inoculate six separate flasks of enriched media to be grown for 100 hours at which time a glycerol stock was made before analyzing for CoQ₁₀ production. After extraction, the best CoQ₁₀ producing strain was determined by ¹H NMR and TLC spot intensity under UV light and I₂ staining. The glycerol stock of the best CoQ₁₀ producing strain was used to streak an agar plate for the next round of species purification. After three rounds of selection, Sporidiobolus johnsonii was independently verified by ribosomal sequencing by SeqWright (Houston, Tex.) and Microcheck (Northfield, Vt.). Additionally, the strain matched physical descriptions of known strains of S. johnsonii, as shown in FIGS. 3 and 4A-C.

Development of Sporidiobolus johnsonii Minimal Media (“SjMM”)

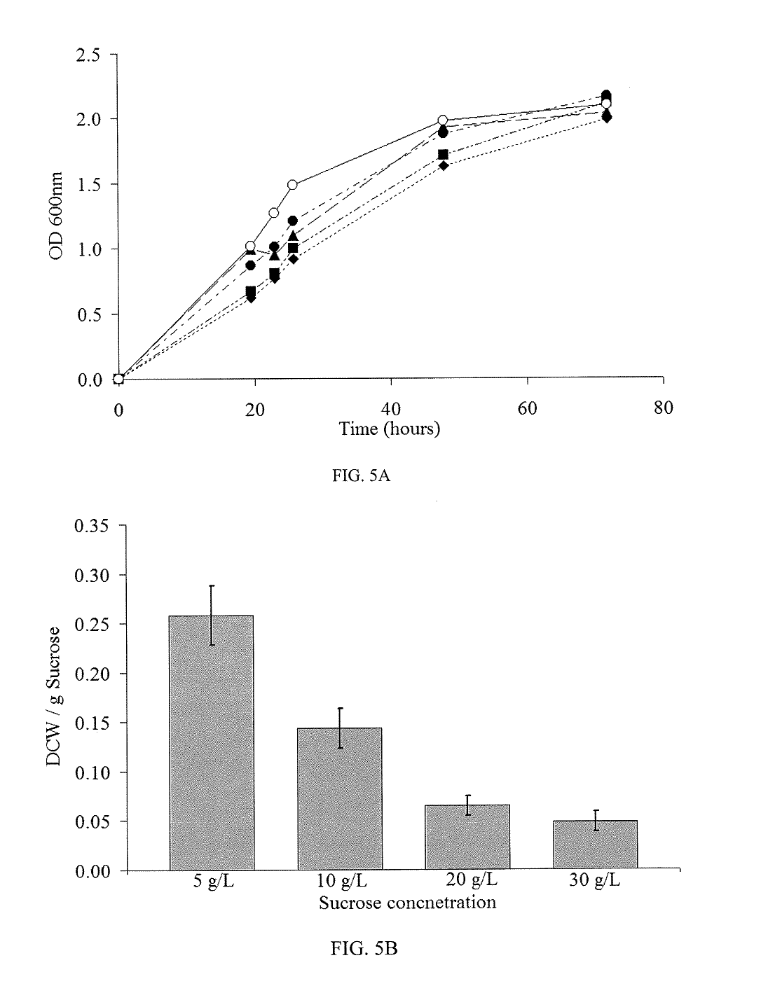

In order to properly investigate the effect of addition of a single media component, a minimal media was developed based upon yeast nitrogen base, but reduced to a single nitrogen, phosphate, and carbon source with a minimal number of metal salts (27 μM CaCl₂, 4 mM MgCl₂, and 100 μM FeCl₃) without further optimization. MES (50 mM) buffer was used to control pH to 6. Although a minimal media is expected to permit less cell growth, any insight gained in the SjMM should be applicable to an enriched media. A less complex media is also better to assess the effect of an added ingredient. Sucrose was chosen as the carbon source due to better cell growth, data not shown. The concentration of sucrose was kept low as it provided the most efficient use of the carbon as shown in FIGS. 5A and 5B. At the lowest concentration of sucrose tested, 1.29 g L⁻¹ DCW was produced, giving a 26% efficiency in the use of sucrose at 5 g This was confirmed in a 1 L fermentation to give 1.37 g L⁻¹ DCW or 27% efficient use of sucrose at 5 g L⁻¹. At higher sucrose concentrations, there is a less efficient utilization of the carbon. The percent utilization of carbon appears to be inversely proportional to the initial concentration of sucrose. It is desirable to have the yeast consume as much of the sucrose as possible, because high sucrose levels have been shown to inhibit CoQ₁₀ production. This SjMM provided adequate growth with a lag time of around 20 hours and a doubling time of 14 hours to reach stationary phase by 60 hours with a final OD of approximately 2 and a DCW of 1.3 g/L in 50 mL shake cultures and 1 L fermentation. For comparison, the enriched media gave a final OD of 2.6 and a DCW of 3.2 g/L. Although the SjMM produces a lesser cell density, any insights gained should be applicable to an enriched media system.

Development of Lysis and Extraction Methods

The choice of solvent for the extraction of CoQ₁₀ is important as it must be able to remove the CoQ₁₀ from within the mitochondrial membrane. The solvent, in combination with sonication, aids in cell lysis. The presence of water has been shown to inhibit extraction of hydrophobic compounds. In order to investigate this, 1 g samples of wet cells were either extracted wet or lyophilized to dryness and then extracted once with 20% methanol in dichloromethane with sonication. After filtering and concentration, the extraction efficiency was compared based upon the mass extracted divided by what the DCW would have been had the cells been dry. Results are shown in Table 1. The wet cells gave an extract equal to 69% of the mass of the DCW, compared to 2.6% for the extraction from dry cells. It was apparent that all the mass was not the desired extract. In order to account for any residual water or hexanes insoluble compounds, the extract was redissolved in hexanes, filtered, and analyzed by UV-Vis giving an Abs₂₇₅ₙₘ=0.08 and 1.11 for the extract from wet and dry cells respectively. Evaporation of the solvent gave a mass representing 0.27% of the DCW for the wet cell extraction compared to 1.3% for the dried cell extraction. The larger absorbance as well as the larger mass of extract after redissolving in hexanes suggests that drying process of the cells is important for proper extraction.

Table 1 below shows extraction efficiency as a function of water content shows that extraction efficiency increases with decreased water content. Values are an average of three trials with errors representing one standard deviation.

TABLE 1
Extraction efficiency of wet and dry cells

| | Wet cells | Dry cells |
|---|---|---|
| CH₃OH/CH₂Cl₂ | 69 +/− 7 | 2.6 +/− 0.1 |
| Hexanes | 0.27 +/− 0.10 | 1.3 +/− 0.3 |
| Abs_{275 nm} | 0.08 | 1.10 |

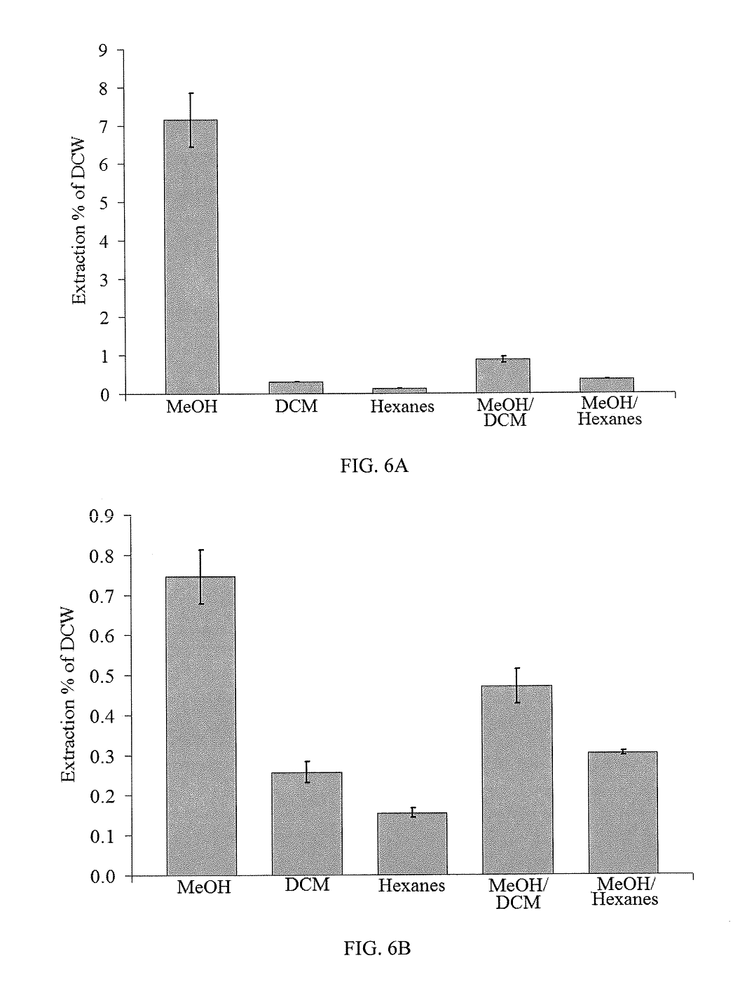

After establishing that dried cells result in better extraction efficiency, it was necessary to determine the best solvent to extract the CoQ₁₀ from the cells. While it has previously been shown that a mixed solvent system provides the optimum extraction of hydrophobic compounds from dried yeast cells, this study did not include an alcoholic solvent. A variety of organic solvents were tested for this purpose. Dried cells were extracted with different types of solvents, alcohol (methanol), polar organic (dichloromethane, DCM), and non-polar organic (hexanes), as well as mixtures thereof. Lysis of 1 g dried cells was performed in 5 mL of the organic solvent with sonication. The ratio of solvent to cell mass was kept intentionally low so as to exaggerate the difference in extraction efficiencies, shown in FIGS. 6A and 6B. It was discovered that the methanol extracted the largest amount of material and also increased the extraction efficiency when added to dichloromethane or hexanes. The dichloromethane was better for extraction compared to hexanes when used individually, but the difference was greater when methanol was used to aid extraction efficiency. Thus the overall trend was that the more polar organic solvent was able to extract more mass. The evaporated extract was extracted with hexanes until the hexanes were clear. ¹H NMR and TLC analysis (data not shown) of the remaining residue indicates no CoQ₁₀ present in the residue remaining in the non-hexanes soluble portion. The hexanes soluble portion from the methanol extract still gave the best greatest mass and CoQ₁₀ was confirmed by ¹H NMR. The overall results suggest that methanol is the most efficient solvent for extraction compared to dichloromethane and hexanes. This is most likely due to inability of the nonpolar solvent to get to the mitochondria through the cytoplasm and hydrophilic surface proteins. The methanol is able to dissolve these and get to the mitochondria for the CoQ₁₀. The results also indicate that an initial solvent based purification can be done by resuspending the methanol extract in hexanes and filtering, with no CoQ₁₀ left behind.

Investigation of CoQ₁₀ Production

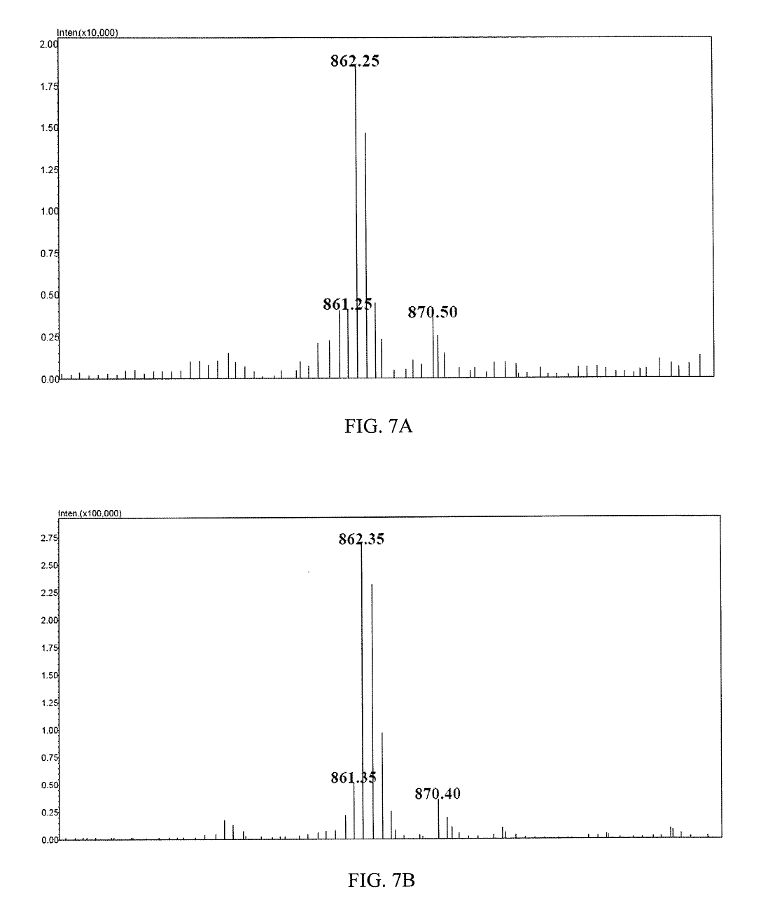

Due to the scarcity of literature relating CoQ₁₀ production to S. johnsonii, it was necessary to prove that this strain of S. johnsonii produces CoQ₁₀ as the major ubiquinone. For this purpose, S. johnsonii was grown in enriched media (“SjEM”). Initially, the crude extract was developed on silica gel TLC using 15% diethyl ether in hexanes to give a spot with Rf=0.3 which corresponded with the standard CoQ₁₀. The spot was scraped, eluted with diethyl ether, evaporated under reduced pressure, and redissolved in methanol for APCI-MS analysis. The mass obtained was 862.25 amu, which correlates well with the mass of 862.35 amu obtained from the CoQ₁₀ standard, as shown in FIGS. 7A and 7B.

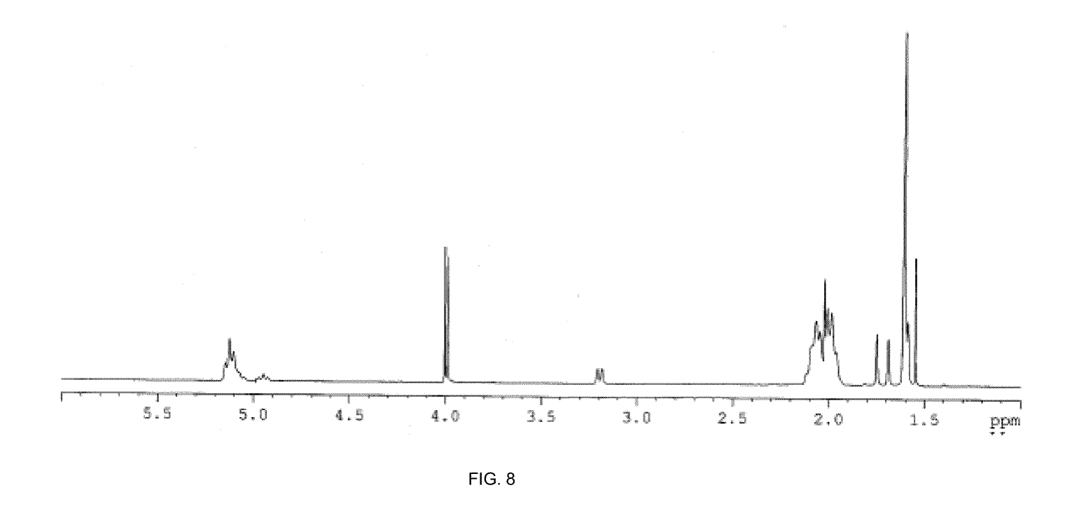

Prep TLC premitted purification of a larger amount of product, giving enough sample for ¹H NMR analysis. The ¹H NMR, shown in FIG. 8, matches the ¹H NMR of commercial CoQ₁₀ standard. Full spectra available in supplemental information. The only difference is the relative intensity of the water peak seen at 1.55 ppm. The electronic absorption spectra showed the expected absorbances at 210 and 275 nm with the molar extinction coefficient, ε₂₇₅, calculated as 13,000 M⁻¹ cm⁻¹. The experimentally determined ε₂₇₅ for the standard was 15,700 M⁻¹ cm⁻¹.

Analysis of a Major Impurity

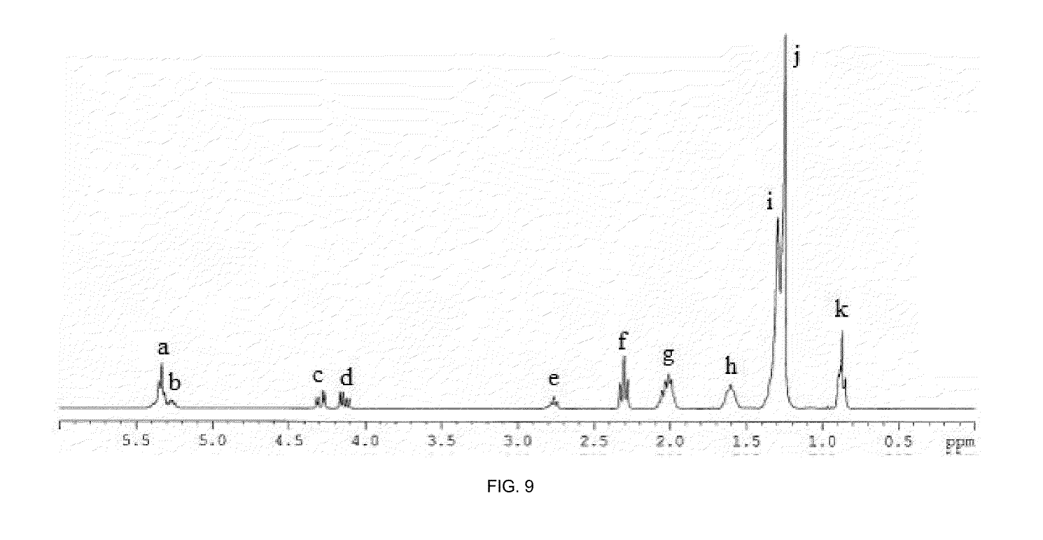

During the process of confirming the identity of CoQ₁₀, a relatively non-UV active compound was discovered to elute close to the CoQ₁₀ in a streaking manner, as shown in FIG. 9, making purification difficult.

The purified compound was analyzed by ¹H NMR, ¹³C NMR, ³¹P NMR, UV-Vis, MS, and elemental analysis, all of which suggested a triacyl glycerol (“TAG”). The ¹H NMR (CDCl₃), shown in FIG. 9, was compared to standard ¹H NMR spectra, and shows the presence of a glycerol unit indicated by the pair of doublet of doublets at 4.1 ppm and 4.3 ppm, labeled as ‘c’ and ‘d’, along with the partially hidden multiplet at 5.2 ppm labeled as ‘b’. Further analysis of the ¹H NMR spectra reveals 8 olefinic protons at 5.3 ppm labeled as ‘a’ indicating the presence of four C—C double bonds. The multiplet at 2.7 ppm, labeled as ‘e’, represents two genimal protons with a C—C double bond on each side. The pseudo triplet at 2.3 ppm, labeled T, represents the α-CH₂ group on each fatty acid (FA) chain and integrates for six protons indicating the presence of three fatty acid esters with saturated α-CH₂ groups. The multiplet at 2.0 ppm, labeled ‘g’, is from six different CH₂ groups (12H) adjacent to an olefin. The peaks at 1.6 ppm, labeled ‘h’, represent the fully saturated β-CH₂ group on each of the three FA esters. The peaks at 0.9 ppm, labeled as ‘k’, represent the terminal methyl groups on each of the three FA, and integrates for nine indicating that there is no double bond in the ω or ω-1 positions. The peaks labeled as ‘I’ and ‘j’ at 1.3 and 1.2 ppm are from the plain CH₂ groups on the FA chain.

The ¹³C NMR correlates well with the NMR. Three signals appear around 173 ppm indicating the carbonyls from the fatty acid group. The signals at 69 and 62 ppm are from the glycerol unit. Three peaks are expected, but it appears that the 62 ppm peak represents 2 carbons. There are 8 signals in the olefin region around 130 ppm indicating 4 carbon-carbon double bonds. Olefins in the a or (3 positions have been shown to have ¹³C peaks at 119/152 ppm and 120/134 ppm respectively. The absence of signals near 120 ppm supports lack of a double bond on the α or β carbon which correlates with the ¹H NMR. This combined data suggests a TAG with four olefins which are not on the α, β, or ω positions, but still has not given any indication as to their distribution within the three fatty acid groups.

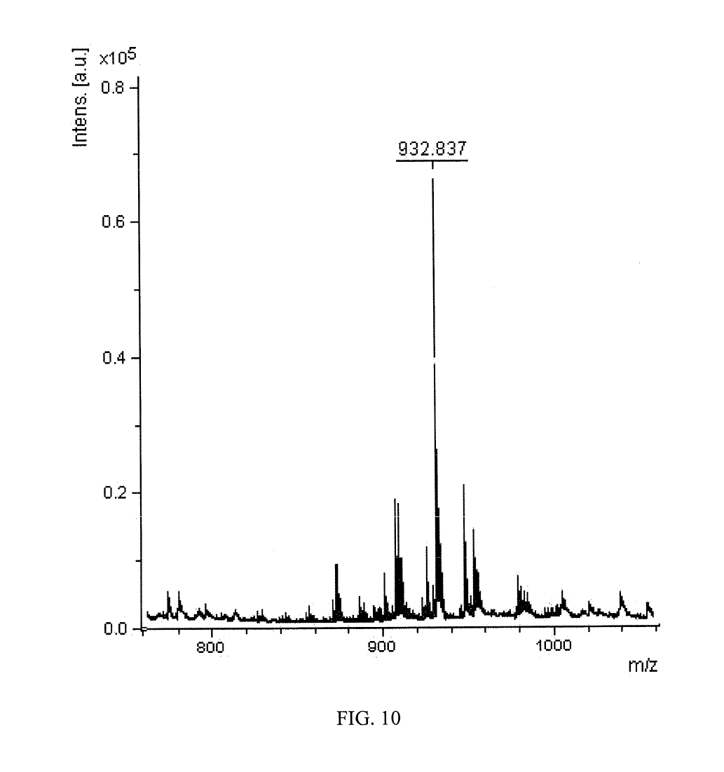

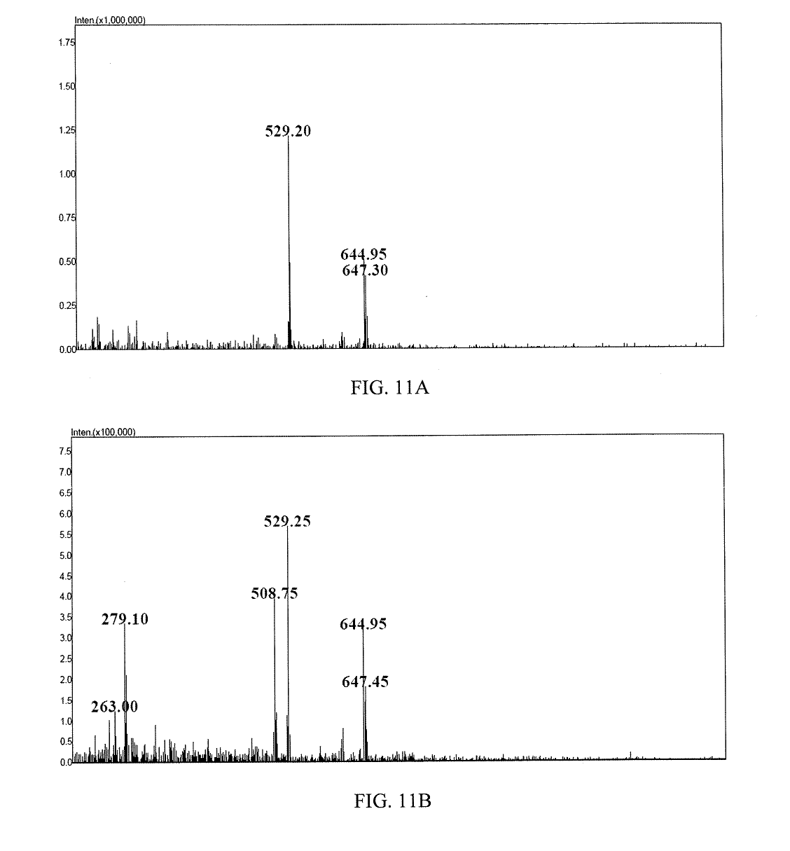

The MALDI-MS detected an ion at m/z=933 shown in FIG. 10, most likely the sodium adduct as has been shown to be dominant over M⁺ or [M+H]⁺, which gives an actual molecular mass of 910 amu. Additional fragmentation could not be conclusively identified in the MALDI due to matrix background. Combining this with elemental analysis data, the molecular formula of C₅₉H₁₀₆O₆ was calculated, which fits the numbers of protons counted in the NMR. The UV-Vis spectral data also suggests a TAG by the absorbance maximas as well as the molar extinction coefficient determined to be 35 M⁻¹ cm⁻¹, which is in line with other TAGs having low extinction coefficients consistent with non-conjugated double bonds.

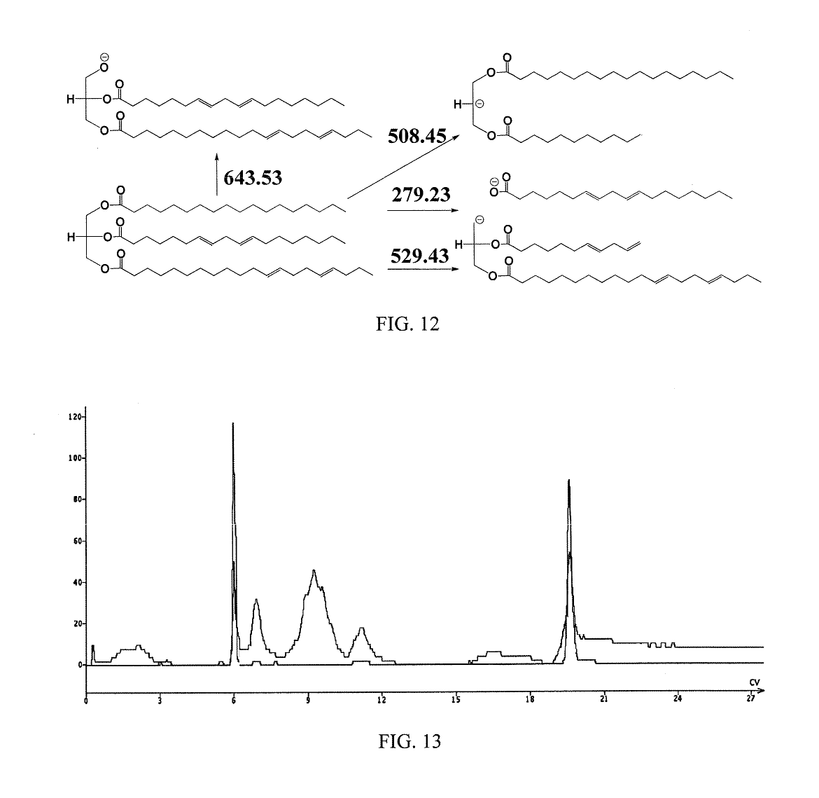

Additional MS fragments can be seen in the APCI-MS data, see FIGS. 11A and 11B, showing prominent peaks at m/z=529 and 645 amu. Upon higher energy CoQ₁₀ y ionization, additional fragments can be seen at m/z=279 and 509 amu. The 645 amu ion results from loss of one FA group with a mass of 279 which corresponds to a FA of the 18:0 type of a formula of C₁₈H₃₈O₂. The other major fragment detected at 529 amu may provide evidence for the position of the double bonds in the other FA chains. This mass of 529 may be additional fragmentation from the 645 amu fragment. This suggests loss of the 18:2 FA with the loss of an additional C₈H₁₇ fragment, thereby suggesting that there are two double bonds on the non 18:0 FA portion which is ionized off. Upon additional ionization energy, a fragment at 508 amu is detected stemming from loss of a FA of the 18:2 type and additional fragmentation to lose another fragment of C₉H₁₅. This additional loss comes from the other FA and indicates that a double bond is located at position C12-C13 with another double bond between C15-C18. APCI-MS has thus provided additional fragmentation giving insights into the nature of the FA esters as far as number of C—C double bonds and their approximate locations, see FIG. 12.

Purification of CoQ₁₀

In cells, CoQ₁₀ is embedded into lipid membranes made up of di- and triglycerols, DAGs and TAGs. It is then no surprise that the TAG and CoQ₁₀ tend to stay together during normal phase chromatography as they have preferential hydrophobic interactions. It was thought that reverse phase liquid chromatography (RPLC) on C18 stationary phase should provide enough hydrophobicity to resolve the TAG and CoQ₁₀ peaks. Using standard water and acetonitrile mobile phase was unsuccessful as a result of the poor aqueous solubility of CoQ₁₀. While others have used RPLC to purify CoQ₁₀ from fermentation extracts, it was felt these methods were not well suited for long term commercial scale production. As recourse, normal phase silica chromatography was reevaluated. This method was also difficult due to the presence of the TAG in all solvent systems tested, including acetone, diethyl ether, ethyl acetate, methanol, isopropanol, and toluene (all in hexanes). Diethyl ether turned out to be the best solvent to separate CoQ₁₀ from the closely eluting impurity; however acetone gave similar results and is less hazardous and was therefore chosen for purification. In order to provide a more reproducible purification method, a Biotage Isolera Flash Chromatography system was used with a 4 g silica cartridge (Teledyne Redi-Sep Gold Rf).

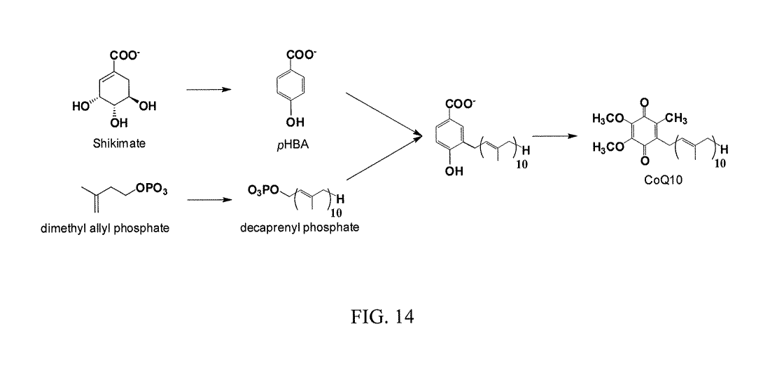

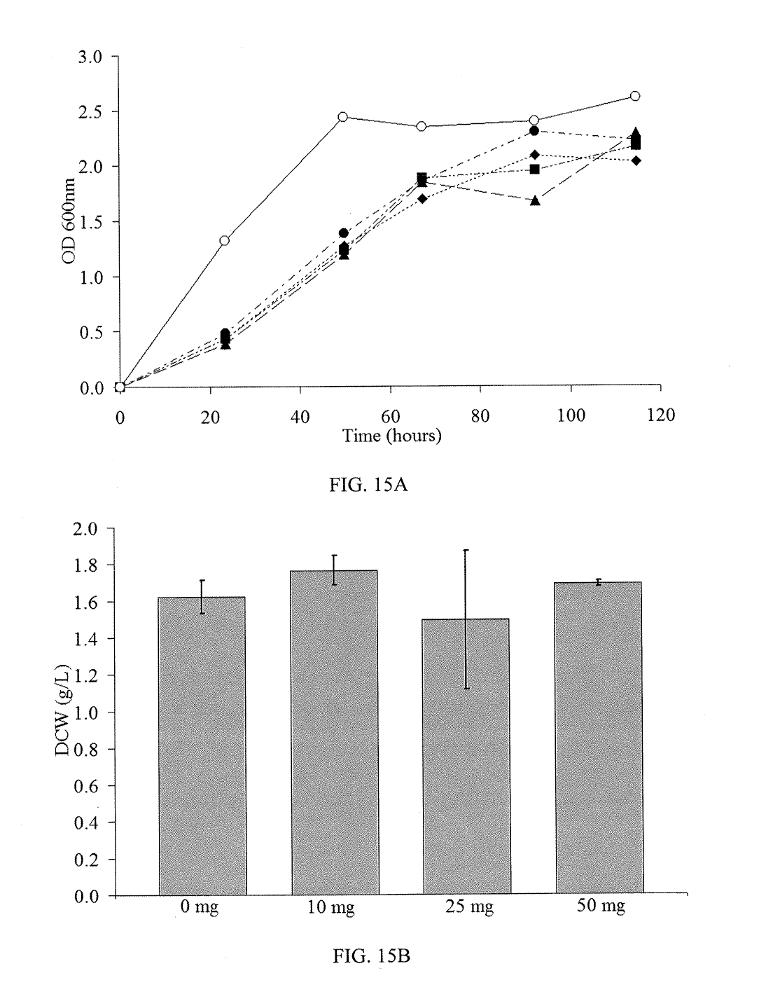

The final purification method resulted in the trace shown in FIG. 13 and included hexanes as solvent A, and acetone as solvent B in the following gradient: 0% B for 3 column volumes (CV), 0-15% B over 5 CV, 15-20% B over 15 CV, 15-80% B over 5 CV, followed by 80% B for 5 CV. This method was successful at purifying CoQ₁₀ from the crude fermentation mixture as confirmed by ¹H NMR (shown in FIG. 6B above). In an attempt to shorten the method, the initial isocratic hexanes run was eliminated, however it was found that this did not provide adequate resolution.

Effects of Added Hydroxy Benzoic Acid (“HBA”)

Selection of added media components to aid in CoQ₁₀ production can be aided by looking at the biosynthesis of CoQ₁₀ in yeasts. FIG. 14 shows a general biosynthetic pathway for CoQ₁₀ production. CoQ₁₀ is produced by joining an aromatic precursor, para-hydroxy benzoic acid (“HBA”) (also known as 4-Hydroxybenzoic acid or p-hydroxybenzoic acid), to a decaprenyl diphosphate group followed by subsequent ring modification. HBA is an interesting choice as it is the first dedicated intermediate towards CoQ₁₀ biosynthesis and there are no pathways competing for HBA.

The presence of HBA during log growth phase stunted cell growth. This was resolved by addition of HBA at 60 hours, after log growth phase, resulting in a DCW indistinguishable from that with no HBA. The later addition of HBA should not be detrimental to CoQ₁₀ production as CoQ₁₀ is not produced in significant quantities until well into the stationary phase. FIGS. 15A and 15B show the growth profile and DCW yields as a function of amount HBA added. There was no statistical difference in DCW in the concentration range of HBA tested in the 50 mL shake flasks, all giving around 1.6 g L⁻¹. The 1 L fermenter gave 2.3 g L⁻¹ DCW. This increase in DCW is likely a result of controlling pH with 5% acetic acid, an additional carbon source. The 50 mL shake flasks never turned basic to require the addition of acid to control pH.

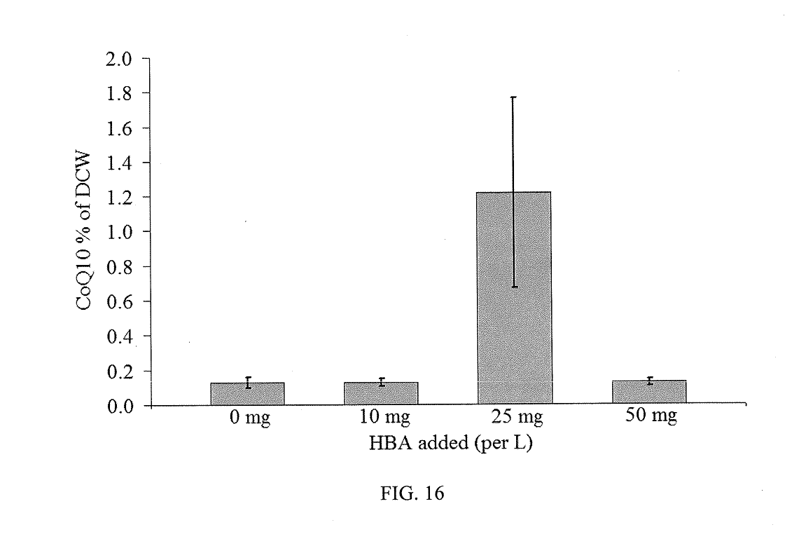

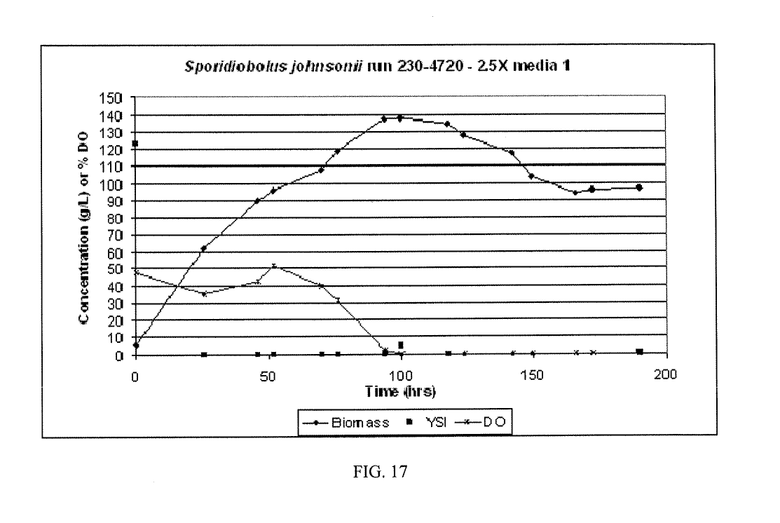

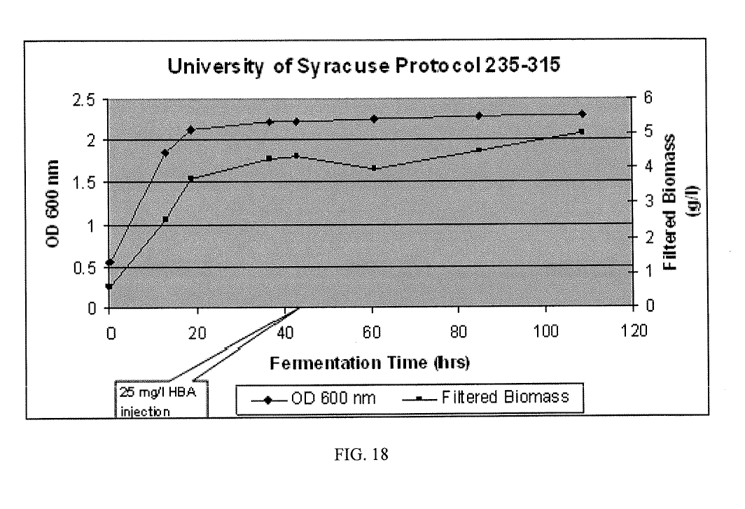

While HBA may inhibit cellular growth when present from the initial inoculation, it bears no consequence on DCW when added after log growth phase. The concentrations tested included 0 mg (0 mM), 10 mg/L (12 mM), 25 mg/L (29 mM), and 50 mg/L (58 mM) and results are shown in FIG. 16. The control with no HBA added produced very little CoQ₁₀, 0.13%. The lowest concentration of HBA added, 10 mg/L, provided a statistically insignificant increase in CoQ₁₀ production. The middle concentration of HBA tested, 25 mg/L, gave the optimum CoQ₁₀ production at statistically significant levels, 1.2%. The most concentrated HBA culture gave a CoQ₁₀ production similar to the culture with no HBA addition.

The negligible increase in CoQ₁₀ production in the 10 mg/L HBA culture was unexpected, but most likely results from increased, yet inadequate stimulation of the enzymatic machinery necessary to convert the HBA to CoQ₁₀. The low CoQ₁₀ production in the most concentrated HBA culture was also a surprise. It was anticipated that specific CoQ₁₀ production would be increased but with a greater effect being a decreased DCW which was not observed. The optimal HBA concentration was determined to be 25 mg/L which resulted in a CoQ₁₀ yield of 1.2%. This is a highly significant increased CoQ₁₀ production as a result of added HBA specifically after log growth phase.

While HBA was used in these studies, it remains a possibility that other biosynthetic precursors of CoQ₁₀ production could be used to encourage CoQ₁₀ production in the Sporidiobolus johnsonii strain, as would be recognized by one of ordinary skill in the art. Such precursors could include 3-methyl-3-buten-1-ol, vegetable juices, or tobacco leaf, for example.

This newly isolated yeast has been ribosomally sequenced and identified as Sporidiobolus johnsonii. This is the first quantification of CoQ₁₀ production in any strain of S. johnsonii. The base production of CoQ₁₀ in SjMM was, 0.13%, among the higher CoQ₁₀ producing yeasts. Upon addition of exogenous HBA, the specific CoQ₁₀ production was increased by nearly an order of magnitude, to 1.2%. This CoQ₁₀ yield makes this strain of S. johnsonii a potential candidate for commercial production of CoQ₁₀. It has also been determined that optimal CoQ₁₀ recovery only occurs with dried cells extracted with an alcoholic solvent. Hexanes can be used as an initial solvent based purification to remove much of the unwanted material. A standardized purification method has been developed on a fully scalable instrument using hexanes and acetone. Thus the entire process does not expose the cells or CoQ₁₀ to any chlorinated solvents. With a complete process in place for a strain of S. johnsonii capable of producing over 1% CoQ₁₀, future pilot scale fermentations are being investigated.

CoQ₁₀ Production by Large-Scale Fermentation

After initial bench top data showed potential for high specific CoQ₁₀ production, it was desired to maximize the cell density in order to maximize the overall CoQ₁₀ yield in mg CoQ₁₀/L. This was accomplished in collaboration with the Iogen Corporation, Ottawa, Canada, and several 14 L fermentations using conditions optimized for cell growth were conducted.

The conditions used to promote cell growth included using a very enriched corn steep liquor (“CSL”) feed source, a high dissolved oxygen (“DO”) concentration of 40-50%, and operating under a batch-fed method. The batch-fed method consists of feeding additional sugar when the sugar in the media had been consumed. After the lag phase, the sugar content is near zero due to the rapid consumption by the yeast in addition to the addition of a minimal amount of sugar once stationary phase is reached. The pH was maintained at 6 with 30% NH₄OH. The highest cell density achieved was 140 g DCW/L (see FIGS. 15A and 15B). This high cell density is extremely good for yeasts, with a cell density of 40-80 g DCW/L normally considered a high cell density for many yeasts and very high cell densities obtained, 95-110 g DCW/L, can be obtained in yeasts such as Saccharomyces cerevisiae. Considering the fact that S. johnsonii is a relatively unknown species, this makes the high cell density achieved of even greater significance. The combination of these two theoretical highs, 10.5 mg CoQ₁₀/g DCW and 140 g DCW/L, provide a maximal theoretical high of 1.47 g CoQ₁₀/L.

With the establishment of the theoretical high values of specific CoQ₁₀ production and cell density, the goal was then to combine the conditions in order to achieve as close to the two theoretical high values simultaneously. The major hurdle in this, other than non-linearity in scaling up, is that the conditions providing the two high values are not compatible. The high CoQ₁₀ production was achieved in nutrient limiting conditions, while the high cell mass was produced in nutrient rich conditions.

As an initial validation of bench top results, a 14 L fermentation was run using the SjMM (see FIG. 16) and produced around 5.1 g DCW/L, more than the 1.7 g/L obtained in bench top conditions with less control over dissolved oxygen. The effect of HBA was investigated using the optimal value determined of 25 mg HBA/L (see FIG. 13), although concentrations up to 1.5 g/L of HBA were used. This 14 L scale fermentation using SjMM produced 3.9 mg CoQ₁₀/g DCW. This result fits well with the bench top data considering that this run gave 2.9 times the concentration of cells and the factor difference in specific CoQ₁₀ yield is 2.7. This shows that there is agreement between the bench top data and the pilot scale fermentation.

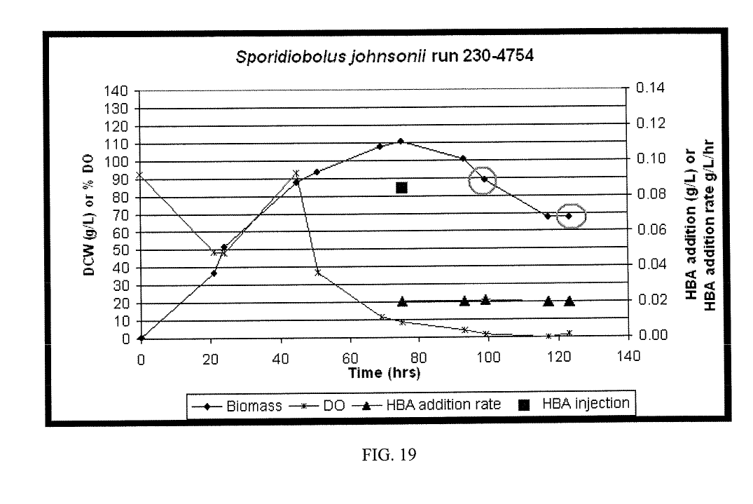

FIG. 19 shows Iogen data from a 14 L fermentation using CSL media with a bolus HBA addition of 0.085 g/L at 78 hrs followed by a steady addition of 0.02 g HBA/hr. The first time point indicated with a circle gave 0.2 mg CoQ₁₀/L with a DCW of 90 g/L resulting in 18 mg CoQ₁₀/L. The specific CoQ₁₀ yield at the final time point circled was up to 3 mg CoQ₁₀/g DCW of 69 g/L resulting in 207 mg CoQ₁₀/L. The cell death and increased CoQ₁₀ production at the final time point is partially a result of the limited nutrients and limited DO, but primarily due to the addition of HBA and its related toxicity. Subsequent fermentations (described below) focused on preventing excessive cell death at the end of the fermentations as well as going to longer fermentation times.

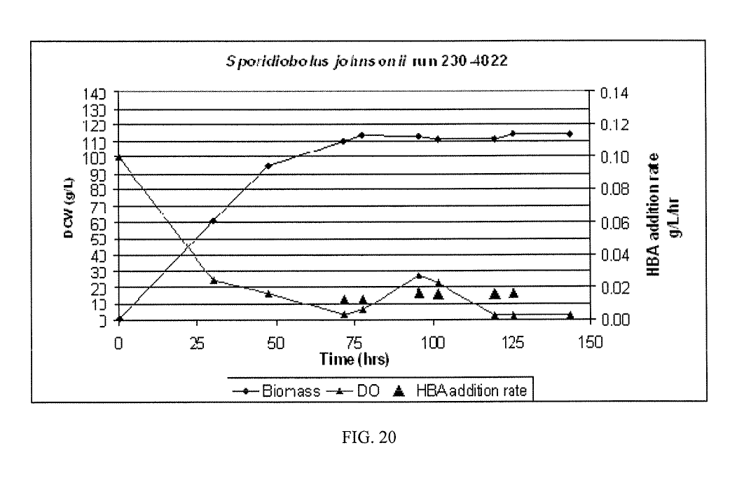

The data in FIG. 20 is from a 14 L fermentation with a very low but steady HBA feed of 0.10 g/L/hr starting at 73 hrs and increasing to 0.15 g/L/hr at 96 hrs. There was no bolus injection of HBA in this run. The HBA feed was kept low in order to prevent cell death. The problem of excessive cell death was avoided in this run. The first data point indicated with a circle produced 0.3 mg CoQ₁₀/g DCW at 113 g DCW to give 34 mg CoQ₁₀/L at 96 hrs. The middle data point indicated produced 0.7 mg CoQ₁₀/g DCW at a density of 115 g DCW giving 81 mg CoQ₁₀/L at 126 hrs. The final time point produced 2.0 mg CoQ₁₀/g DCW at a density of 115 g DCW providing 229 mg CoQ₁₀/L. This fermentation demonstrated that the problem of cell death can be overcome by slower HBA feeding. Also, a lower specific CoQ₁₀ yield is observed at the lower HBA feeding. Thus a high level of HBA is required for high specific yields of CoQ₁₀, and lower levels of HBA are required for higher yields of cells density.

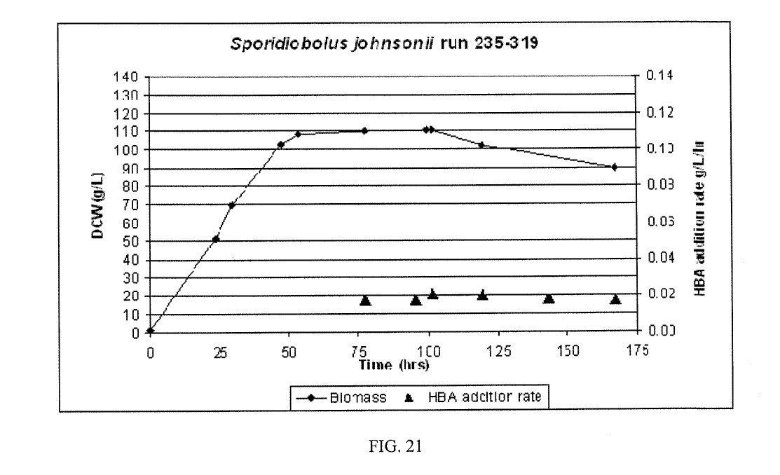

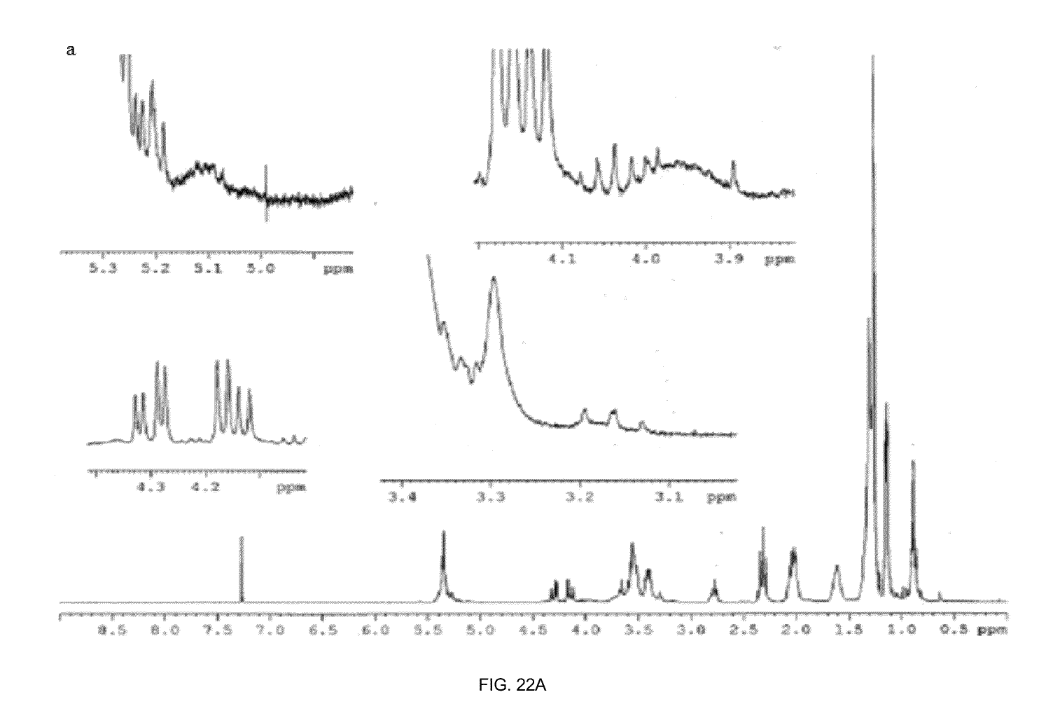

The data in FIG. 21 was obtained with the goal of a low HBA feed and longer fermentation times to maximize specific CoQ₁₀ production. The first data point indicated produced 0.7 mg CoQ₁₀/g DCW at 110 g DCW providing 77 mg CoQ₁₀/L. The second time point indicated produced 1.1 mg CoQ₁₀/g DCW at 104 g DCW providing 114 mg CoQ₁₀/L at 120 hrs. The third data point indicated at 144 hrs produced 1.7 mg CoQ₁₀/g DCW at 96 g DCW providing 163 mg CoQ₁₀/L. The final time point at 168 hrs produced 3.1 mg CoQ₁₀/g DCW at 90 g DCW providing 279 mg CoQ₁₀/L.

The data shown in FIGS. 19-21 represent the relationship between HBA, specific CoQ₁₀ yields, and cell viability. FIG. 19 shows results of a large bolus injection that resulted in significant cell death (69 g final DCW compared to 110 g DCW prior to HBA addition), but a large increase in specific CoQ₁₀ production (3 mg/g DCW) resulting in an overall yield of 207 mg CoQ₁₀/L. FIG. 20 shows that by eliminating the bolus addition of HBA, the problem with cell viability is virtually eliminated. The specific yield of CoQ₁₀ is lower, but still significant at 2 mg/g DCW, resulting in an overall CoQ₁₀ yield of 229 mg/L. The data set in FIG. 21 demonstrates that elimination of the bolus HBA addition, but an increased HBA addition rate (0.02 vs 0.01 g/L/hr) will result in the expected cell viability (90 g final DCW from 110 g DCW prior to HBA addition), much more viable compared to the bolus HBA addition in FIG. 19 (69 g final DCW), and a little worse than FIG. 20 (115 g final DCW). However, the higher specific CoQ₁₀ yield (3 mg/g DCW) provided the highest overall CoQ₁₀ yield of 279 mg/L. This data shows that by optimizing the HBA addition, the final CoQ₁₀ yield can be maximized, but at the expense of one of the two values of cell density or specific CoQ₁₀ yield.

This final result of 3.1 mg CoQ₁₀/g DCW is a very significant result, since it indicates the great potential of this organism for CoQ₁₀ production. A major factor in the CoQ₁₀ production here is the strategic use of a dedicated biosynthetic intermediate, HBA, in order to increase the specific production of the end product. The result demonstrates that knowledge of the use of CoQ₁₀ within the host can help obtain higher specific yields within that host. CoQ₁₀ is used for energy production, primarily after all other sources of energy are exhausted, usually late into the stationary phase. This is highlighted by the increased specific yield at the later time point, 175 hours producing 3.1 mg CoQ₁₀ g DCW, compared to earlier time points of 140 hours producing 2 mg CoQ₁₀/g DCW.

Cell Drying Methods

The promising pilot scale fermentation data required investigation into industrial scale methods of drying the cells prior to lysis and extraction. Two drying methods were investigated, cyclone and blow down. Both methods began with the cells being resuspended in a minimal amount of water at a particular pH, usually basic. The cell slurry was then pumped through pipes heated to temperatures up to 185° F. (85° C.) and then to the collection chamber. For cyclone collection, the cell suspension was pumped into a heated spinning cylindrical barrel with porous walls. The cells were then pressed against the wall and the centripetal force of the spinning caused the moisture to go through the porous walls, with dry cells being left. In the blow down collection method, the cell slurry was pumped through a small heated opening at an extremely high velocity into a porous bag. The very high velocity and heat caused the moisture to go through the bag and evaporate. Both methods were investigated.

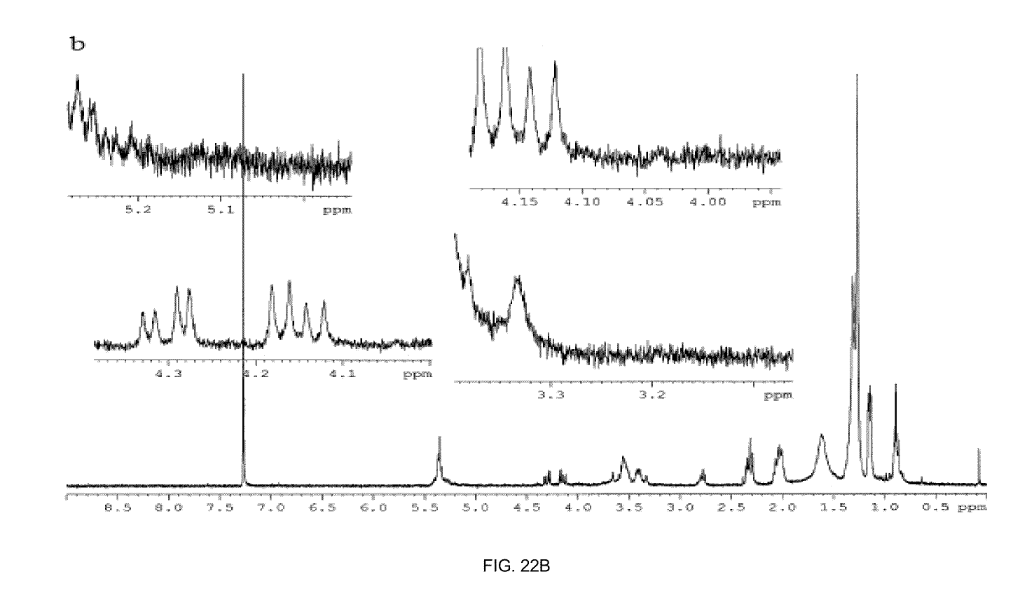

Cells from a pilot scale fermentation were shipped to Pulse Combustion Systems (Texas), who dried a portion of the cells by each method and then shipped the dried cells here to Syracuse University. There was an obvious color difference between the two sets of dried cells, with the blow down cells being darker with a greater degree of brown rather than pink coloration. Each set was extracted using 20% DCM in hexanes for a total of five extractions. The sample from cyclone dried cells was the expected orange color, while the sample from the blow down cells was much less intense in color and appearing more pink rather than orange. After evaporation of solvent, the residue was analyzed by ¹H NMR (see FIGS. 22A and 22B).

The NMR's in FIGS. 22A and 22B show that the CoQ₁₀ decomposes in the blow down drying method. This is evidenced by the presence of CoQ₁₀ peaks in the NMR from the cyclone sample but not the blow down method. The CoQ₁₀ peaks of interest are at 5.1 ppm, 4.0 ppm, and 3.2 ppm. There are peaks in all three of these areas in the cyclone sample (FIG. 22A) albeit in the presence of additional peaks. The NMR from the blow down cells (FIG. 22B) do not show any of these peaks. Both drying methods utilize high temperatures (65-85° C.), but the cyclone method is gentler on the cells. It is possible that the kinetic energy (in addition to heat) involved in the blow down process may be enough to cause decomposition of the CoQ₁₀.

Materials and Methods

Chemicals

Chemicals used in connection with the present invention comprise Malt extract and CaCl₂.2H₂O were purchased from EMD. NH₄OH (30%) and KH₂PO₄ were purchased from BDH. Sucrose and FeCl₃.6H₂O were purchased from Sigma. NH₄Cl was from Fisher. MgCl₂.6H₂O was purchased from Acros. Yeast extract was purchased from BP/Bacto. para-hydroxy benzoic acid (HBA) was purchased from Alfa Aesar. Antifoam C was purchased from JT Baker. Hexanes and acetone chromatography solvents were purchased from BDH. CoQ₁₀ standard was provided by PharmaBase (Switzerland).

Media and Culture Conditions

Sporidiobolus johnsonii enriched media (“SjEM”) consisted of 5 g l⁻¹ malt extract, 5 g l⁻¹ yeast extract, 1 g KH₂PO₄ and the pH was adjusted to 5.65 with 1M NaOH followed by a 20 minute autoclave cycle. The Sporidiobolus johnsonii minimal media (SjMM) was composed of 5 g l⁻¹ sucrose, 4 g l⁻¹ NH₄Cl, 1 g l⁻¹ KH₂PO₄, 0.1 g NaCl, 10.6 g MES and then adjusted the pH to 6.0 with 30% NH₄OH. After a 20 minute autoclave cycle, the following salts were aseptically added from a concentrated solution to the following final concentrations, MgCl₂.6H₂O (0.85 g/L, 4 mM), CaCl₂ (0.13 g/L, 0.9 mM), and FeCl₃.6H₂O (0.27 g/L, 1 mM). No pH adjustment was made after addition of the metal salts. After 10% inoculation from a seed culture (described below) the flask was incubated at 30° C. and shaken at 300 RPM. 1 mL aliquots were taken for optical density (OD) measurements at 600 nm on a Varian Cary 50 Bio UV-Vis spectrophotometer. The pH was measured on a Mettler Toledo SG2 pH meter and adjusted as needed with NH₄OH.

The seed train used for all experiments started with a SjMM agar plate previously incubated at room temperature for two days, from which a single colony was used to inoculate 5 ml SjMM in a 12 ml culture tube. After two days at 30° C. and 300 RPM, all 5 ml were transferred to 50 ml SjMM in a 125 ml flask. Upon overnight incubation, 25 mL of this was transferred to 250 mL SjMM in a 1 L flask. After overnight incubation, this was used in 5 ml aliquots to inoculate a set of 50 ml SjMM cultures and 100 mL was used to inoculate the bioreactor. Fermentation data was obtained using a 1.3 l bioreactor (BioFlo110 New Brunswick Scientific) with an initial volume of 800 mL before addition of the 100 mL seed inoculum. Bioreactor media consisted of SjMM without MES buffer and with the addition of 0.5 ml Antifoam C Medical Grade (BP/Bacto). Temperature was maintained at 30° C. by blanket jacket. The pH was maintained at 6.0 with 3% NH₄OH and 10% acetic acid. Dissolved oxygen (DO) was initially set to 50% and controlled by agitation, which ranged from 50-300 RPM. Airflow was 0.5 LPM. Upon addition of HBA, the DO was changed to 5%.

Cell Lysis and Extraction of CoQ₁₀

Cells were centrifuged using a Sorvall Legend RT centrifuged for 15 minutes at 4000 RPM, then resuspended in water and centrifuged (15 min, 4000 rpm) and decanted. The pellet was then lyophilized in a Labconco FreeZone1 lyophilize equipped with a Welch Chester 1402N vacuum pump for 48 hours and weighed on a Mettle Toledo AB54-S analytical balance to +/−0.1 mg. The dried cells were then extracted with different types of solvents, including alcohol (methanol), polar organic (dichloromethane), and non-polar organic (hexanes). Cell lysis was performed in the organic solvent with aid of a Fisher Scientific Sonic Dismembrator Model 100 with a probe tip on setting 5 for intervals of 10 seconds each. The cell slurry was then filtered through a sintered glass funnel to ensure no cell debris is included in the extract. This was repeated with fresh solvent until no peak was detected at 275 nm. Extracts were combined, and evaporated to dryness. Two additional extracts (20% CH₃OH in CH₂Cl₂, then 20% CH₃OH in Hexanes) were performed on the cells and combined, but kept separate from the first set of extracts. This final extract was determined to have no CoQ₁₀ by ¹H NMR and TLC analysis and was therefore not added to the first extract.

Purification of Cell Extract

Silica gel TLC used 15% diethyl ether in hexanes. Visualization included UV illumination and I₂ staining. A Biotage Isolera Flash Chromatography system equipped with a variable wavelength detector set to 210 nm and 275 nm was also used. The sample, typically 10 mg, was dissolved in 1 mL hexanes and loaded onto a Teledyne Gold Rf column (4 g, 4.6 ml column volume (CV)), which has been preequilibrated with hexanes. Mobile phase A consisted of hexanes and phase B was acetone. The gradient was 0% B 1.5 CV, 0-5% B 1.5 CV, 5-15% B 14 CV, 15-80% B 1 CV, 80% B 5 CV at a flow rate of 10 ml min⁻¹.

Characterization of CoQ₁₀ and the Coeluting Impurity

APCI-MS was preformed on a Shimadzu LCMS2010-A set to negative ion detection using 100% acetonitrile as a mobile phase. The interface voltage was set to 3.5 kV. Detector was set to 3 kV. The CDL temperature was 250° C. with N₂ nebulizing gas at 2.5 LPM and no drying gas. ¹H and ¹³C NMR spectra were collected on a 300 MHz Bruker NMR. ³¹P NMR and 2-D NMR spectra were collected on a 500 MHz Bruker NMR. Elemental analysis data by Micro-analysis Inc (Delaware). Electronic absorption spectra (EAS) were recorded on a Varian UV-Vis Spectrophotometer in hexanes. MALDI-MS data was collected on a MALDI in positive detection reflectron mode with an N₂ laser. The matrix was α-cyano-4-hydroxycinnamic acid (CHCA) (10 mg/mL) dissolved in 1:1 water:acetonitrile with 0.1% TFA.

Analysis of CoQ₁₀

TLC (15:85 Et₂O:Hexanes, R_{f}=0.3, Standard R_{f}=0.3). APCI-MS negative mode of the TLC purified extract showed [M]⁻ at m/z=862.25 amu (Standard CoQ₁₀ m/z=862.35 amu). ¹H NMR (300 MHz, CDCl₃) δ 5.12 (m, 10H, HC═C) δ 4.00 (s, 3H, ArOCH₃), 3.99 (s, 3H, ArOCH₃), δ 3.19 (d, 2H, ArCH₂C), δ 2.00 (m, 36H, CH₂), δ 1.74 (s, 3H, ArCH₃), δ 1.69 (s, 3H, terminal CH₃), δ 1.59 (s, 30H, C═C(CH₃)C). Standard CoQ₁₀ was the same. EAS (hexanes) λmax=210 nm, and 275 nm. ε₂₇₅ₙₘ=13,000 M⁻¹ cm⁻¹ (Standard CoQ₁₀ λmax=210 nm, and 275 nm. ε₂₇₅ₙₘ=15,700 M⁻¹ cm⁻¹.

Analysis of the Closely Eluting Impurity

TLC (15:85 Et₂O:Hexanes, R_{f}=0.4). APCI-MS negative mode under the same conditions as CoQ₁₀ showed m/z=645.10, 611.10, 529.30, 508.85, and 279.10 amu. MALDI-MS positive ion detection provided the parent [M⁻+Na]⁺ at m/z=933.10. Elemental analysis by Micro-Analysis INC, (Wilmington, Del.) gave C(77.65%), H(11.41%), N(0.08%). ¹H NMR (300 MHz, CDCl₃) δ 5.33 (m, 8H, HC═CH) δ 5.27 (m, 1H, middle CH on glycerol), δ 4.30 (dd, J=4.3 Hz, 2H, CH₂ on glycerol), δ 4.13 (dd, J=5.9 Hz, 2H, CH₂ on glycerol), δ 2.74 (t, J=5.9 Hz, 2H, C═C—CH₂—C═C), δ 2.30 (m, 6H, a-CH₂), δ 2.00 (m; 12H, C═C—CH₂), δ 1.60 (m, 6H, (3-CH₂), δ 1.27 (m, 58H, CH₂ on fatty acid?), δ 0.89 (m, 9H, terminal CH₃). ¹³C NMR (75 MHz, CDCl₃) δ 173.65, 173.61, 173.20, 130.59, 130.40, 130.39, 130.09, 130.06, 128.47, 128.45, 128.29, 69.26, 62.48, 34.58, 34.44, 34.41, 32.34, 32.32, 31.93, 30.17, 30.11, 30.07, 30.03, 30.01, 29.94, 29.89, 29.79, 29.75, 29.73, 29.68, 29.61, 29.58, 29.51, 29.48, 29.45, 27.62, 27.58, 27.57, 26.02, 25.26, 25.23, 23.09, 22.99, 14.53, 14.49. DEPT-135, HSQC, HMBC, and COSEY NMR data included in supplementary information. EAS (hexanes) λₘₐₓ=272, 281, 293 nm. ε₂₈₁ₙₘ=35 M⁻¹ cm⁻¹.

Although the present invention has been described in connection with a preferred embodiment, it should be understood that modifications, alterations, and additions can be made to the invention without departing from the scope of the invention as defined by the claims.

What is claimed is:

1. A biologically pure culture of Sporidiobolus johnsonii strain Sj0801, or a mutant thereof, which is capable of producing at least about 1% CoQ₁₀ by dry cell weight.

2. A method of producing CoQ₁₀, comprising the steps:
providing a microbe, wherein the microbe comprises a Sporidiobolus johnsonii strain;
culturing the microbe in the presence of a carbon source; and
recovering said CoQ₁₀.

3. The method of claim 2, wherein said Sporidiobolus johnsonii strain is the Sporidiobolus johnsonii strain of claim 1, or a mutant thereof.

4. The method of claim 2, wherein the carbon source is sucrose.

5. The method of claim 2, further comprising the steps of:
drying the cultured microbe; and
lysing the microbe.

6. The method of claim 5, wherein the lysing step comprises lysing said microbe in the presence of a solvent.

7. The method of claim 6, wherein said solvent comprises methanol.

8. The method of claim 2, wherein the step of recovering said CoQ₁₀ comprises the steps of:
obtaining an extract of the cultured microbe;
purifying the CoQ₁₀ from said extract.

9. The method of claim 8, wherein the obtaining step comprises the step of lysing said cultured microbe in the presence of a solvent.

10. The method of claim 8, wherein the purifying step comprises chromatography of said cell extract.

11. The method of claim 2, wherein said microbe is cultured in the presence of a CoQ₁₀ biosynthetic precursor.

12. The method of claim 11, wherein said CoQ₁₀ biosynthetic precursor is 4-Hydroxybenzoic acid.

13. The method of claim 12, wherein said 4-Hydroxybenzoic acid is present at a concentration of about 10 mg/L to about 1.5 g/L.

14. The method of claim 2, wherein said culturing step comprises growing said microbe to at least early stationary phase.

15. The method of claim 2, wherein said microbe is cultured in a minimal medium.

16. A method of producing CoQ₁₀, comprising the steps:
providing a microbe, wherein the microbe comprises a Sporidiobolus johnsonii strain;
inoculating a culture containing a carbon source with said microbe;
culturing said microbe to encourage growth of said microbe;
adding 4-Hydroxybenzoic acid to the culture at some point after said inoculating step; and
recovering said CoQ₁₀.

17. The method of claim 16, wherein said Sporidiobolus johnsonii strain is the Sporidiobolus johnsonii strain of claim 1, or a mutant thereof.

18. The method of claim 16, wherein said adding step comprises the addition of 4-Hydroxybenzoic acid to the culture at a single time point.

19. The method of claim 16, wherein said adding step comprises the continuous addition of 4-Hydroxybenzoic acid to the culture for a period of time.

20. The method of claim 16, wherein said adding step comprises the addition of a first amount of 4-Hydroxybenzoic acid to the culture at a single time point followed by the continuous addition of a second amount of 4-Hydroxybenzoic acid to the culture for a period of time, wherein said first amount is larger than said second amount.
